(19) United States
(12) Patent Application Publication
(10) Pub. No.: US 2007/0297988 A1
(43) Pub. Date: Dec. 27, 2007

(54) Optical probes for in vivo imaging

(76) Inventor: Bin Wu, Sharon, MA (US)

Correspondence Address: Bin WU, 80 Bullard St., Sharon, MA, 02067, US

(21) Appl. No.: 11/706,734

(22) Filed: Feb. 15, 2007

Related U.S. Application Data
(60) Provisional application No. 60/815,177, filed on Jun. 21, 2006.
(60) Provisional application No. 60/830,745, filed on Jul. 14, 2006.

(51) Int. Cl. A61K 49/00; A61K 9/66

(52) U.S. Cl. 424/9.6; 424/455; 977/834

(57) ABSTRACT

Disclosed is an image probing conjugate. The conjugate comprises a nanoparticle and a targeting agent. The nanoparticle comprises a dye which is encapsulated by a functionalized polymer, and the targeting agent is bound to the functional group of the polymer. The nanoparticle provides the conjugate with improved stability and an increased concentration of the dye. Therefore, the conjugate of the invention can be used for probing a small target which otherwise cannot be detected. Bonding the targeting agent to the nanoparticle allows precise image probing from the location where the targeting agent is placed.

### CROSS-REFERENCE TO RELATED APPLICATIONS

This application claims priority to U.S. Provisional Applications 60/815,177 (filed Jun. 21, 2006) and 60/830,745 (filed Jul. 14, 2006).

### FIELD OF THE INVENTION

The invention relates to optical probes for in vivo imaging. More particularly, the invention relates to an image probing conjugate which comprises a nanoparticle of a polymer-encapsulated dye and a targeting agent which agent is bound to the functional group of the polymer.

### BACKGROUND OF THE INVENTION

Conventional methods for imaging human bodies include roentgenography, scintigraphy, ultrasound and magnetic resonance imaging. These imaging methods require the presence of a significant tumor mass for reliable detection and as a result, diagnosis is often delayed. Optical imaging is a promising tool for in vivo monitoring of specific molecular and cellular processes, e.g., gene expression, multiple simultaneous molecular events, progression or regression of cancer, and drug and gene therapy. It uses non-ionizing radiation to detect very small amount of light-absorbing materials in vivo. Optical imaging probes comprising a fluorescent marker and a targeting agent that binds specific tumor molecules or cells can be used as contrast agents in molecular imaging for early detection of cancers.

In mammal bodies, hemoglobin is the principal absorber of visible light, and water and lipids are the principal absorbers of infrared light. They have the lowest absorption coefficients in the red and near-IR (NIR) region of approximately 600-1200 nm. The use of NIR light is ideal for imaging deeper tissues. Imaging in the NIR spectrum maximizes tissue penetration and minimizes auto-fluorescence from non-specific sources.

A contrast agent is usually required in optical imaging. Contrast agents enhance the brightness of the imaging. For in vivo imaging in mammal bodies, NIR dyes are most ideal contrast agents because of deep tissue penetration of the NIR light. Cabocyanine dyes are the most widely used NIR optical probes for imaging tumors in small animals and humans. They have exceptionally high molar absorptivity (typically 10⁵ M⁻¹ cm⁻¹). Among cabocyanine dyes, indocyanine green (ICG) is particularly useful because it has been proved by the U.S. Food and Drug Administration for clinic applications. However, ICG has several disadvantages. First, when administered intravenously, ICG has a plasmatic half-life of 2-4 minutes and shows extensive protein binding. The short circulation time and protein binding prevent it from effective use as a fluorescent probe for molecular imaging. Second, ICG is degraded in aqueous solution and the degradation is accelerated by light and heat. The degradation makes it impossible to prepare a stable ICG bio-conjugate in the aqueous media. Finally, the disulfonate group does not react with bioactive molecules under mild reaction conditions. Consequently, it is difficult to conjugate ICG to antibodies, peptides, proteins and other targeting agents for molecular imaging.

Consequently, several ICG derivatives have been developed. These derivatives are reactive and are more stable in aqueous media. However, the toxicity and biocompatibility of these ICG derivatives have not been fully investigated. Saxena et al (International Journal of Pharmaceutics 278, 2004, 293-301) prepared ICG particles by encapsulating ICG molecules in poly(D,L-lactic-co-glycolic acid), PLGA. The size of these particles was found by the authors to be from 300 to 400 nm. These particles can be potentially usefull in clinic application because both ICG and PLGA are FDA approved materials. However, these particles are not suitable for imaging tumor molecules and cells because they do not contain functional groups on their surfaces to bond with molecular and cell targeting agents.

Thus, there is a need in the art for in vivo optical imaging probes that are long-circulating, non-toxic, non-invasive; target-specific, deep-penetrating, and more biocompatible than current imaging probes and methods.

### SUMMARY OF THE INVENTION

The invention is an image probing conjugate. The conjugate comprises a nanoparticle and a targeting agent. The nanoparticle comprises a dye which is encapsulated by a functionalized polymer, and the targeting agent is bound to the functional group of the polymer. The nanoparticle provides the conjugate with improved stability and an increased concentration of the dye. Therefore, the conjugate of the invention can be used for probing a small target which otherwise cannot be detected. Bonding the targeting agent to the nanoparticle allows precise image probing from the location where the targeting agent is placed.

The invention also provides a method for preparing the conjugate. The method comprises producing a nanoparticle by encapsulating a dye in a functionalized polymer, mixing the nanoparticle with a targeting agent, and bonding the targeting agent to the functional group of the nanoparticle. The nanoparticle can be made by mixing the dye with a functionalized polymer in a solution, encapsulating the dye therewith, and then precipitating the polymer-encapsulated dye nanoparticle. The nanoparticle can also be made by in-situ emulsion or suspension polymerization in the presence of a dye. Suitable emulsion polymerization includes miniemulsion and microemulsion polymerizations. The use of functionalized polymer allows the formation of the nanoparticle that has a reduced particle size and increased stability of the nanoparticles in an aqueous media.

### DETAILED DESCRIPTION OF THE INVENTION

The conjugate of the invention comprises a nanoparticle and a targeting agent, two of which are bound preferably by a covalent bond.

The size of said nanoparticle is from 1 nm to 1,000 nm, preferably from 1 nm to 500 nm. It comprises a dye which is encapsulated by a functionalized polymer, and the targeting agent is bound to the functional group of the polymer. The functionalized polymer contains a functional group preferably selected from the group consisting of carboxylic, amino, hydroxyl, halide, thiol, isocyanate, aldehyde, the like, and mixtures thereof. Suitable functionalized polymers include synthetic and natural polymers. Synthetic functionalized polymers can be made by the polymerization of corresponding functional monomers. They can be homopolymers of a functional monomer, or copolymers of a functional monomer with a different functional monomer or with a non-functional monomer.

Examples of suitable functionalized polymers include functionalized polystyrene, polymethyl methacrylate, polyacrylonitrile, polyacrylic acid, polymethacrylic acid, polyvinyl chloride, polyvinylidene chloride, polyvinylidene fluoride, polylactic acid, polyglycolic acid, polycaprolactone, the like, and mixtures thereof. Examples of suitable natural polymers include gelatin, dextrin, chitosan, hyaluronic acid, cellulose, the like, and the mixture thereof.

Carboxylic functionalized polymers are particularly preferred because the carboxylic functional groups can form covalent amide bonds with suitable targeting agents and meanwhile stabilize the nanoparticle suspension in aqueous media to prevent the particles from agglomerating. Suitable carboxylic groups include carboxylic acids, anhydrides, esters, N-hydroxysuccinimidyl esters, the like, and mixtures thereof. Examples of carboxylic functionalized polymers include poly(acrylonitrile-co-acrylic acid), poly(acrylonitrile-co-methacrylic acid), poly(styrene-co-acrylic acid), poly(styrene-co-methacrylic acid), poly(methyl methacrylate-co-acrylic acid), poly(methyl methacrylate-co-methacrylic acid), poly(lactic acid), poly(glycolic acid), poly(lactic acid-co-glycolic acid), polycaprolactone, polyanhydrides, poly(lactic acid-co-caprolactone), the like, and mixtures thereof. The selection of the functionalized polymer depends on many factors, including the stability of the nanoparticles, the ability of the polymer to encapsulate a high concentration of dye, and the functional group needed to link the nanoparticles with the targeting agent. The selection of the functionalized polymer also depends on where the conjugate is used. For instance, if the conjugate is used in human bodies, the functionalized polymers which are approved by the U.S. or foreign FDA (the Food and Drug Administration), such as polylactic acid, polyglycolic acid, poly(lactic acid-co-gclycolic acid), polycaprolactone, and polyanhydrides are preferred.

Preferred dyes are near-infrared (NIR) having an optical absorption wavelength within the range of 600 nm to 1200 nm. More preferably, the near-infrared dye is selected from the group consisting of indocyanine green, IR-780 iodide, IR-780 perchlorate, IR-27, IR-140, IR-676, IR-676 iodide, IR-746, IR-768 perchlorate, IR-775 chloride, IR-777 perchlorate, IR-780 iodide, IR-780 perchlorate, IR-783, IR 786 iodide, IR-786 perchlorate, IR-792 perchlorate, IR-797 chloride, IR-797 perchlorate, IR-806, IR-813 chloride, IR-813 perchlorate, IR-820, and mixtures thereof.

Many other NIR dyes that are commercially available can also be used in the present invention for preparing NIR nanoparticles. These NIR dyes include but are not limited to Cy5.5, Cy5, Cy7, and Cy7.5 (Amersham Biosciences, Piscataway, N.J.), AlexaFluor 660, AlexaFluor 680, AlexaFluor 700, AlexaFluor 750 (Molecular Probes, Eugene, Oreg.), IRD38 and IRD78(LI-COR, Lincoln, Nebr.), NIR-1 and IC5-OSu (Dojindo, Kumamoto, Japan), FAR-Blue, FAR-Green One, FAR-Green Two, FAR 5.5 (Innosense, Giacosa, Italy), ADS775MI, ADS775MP, ADS775PI, ADS775PP, ADS780HO, ADS790NS, ADS800AT, ADS815EI, ADS821NS, ADS830AT, ADS900AF, ADS1065A, ADS1075A, ADS780WS, ADS785WS, ADS790WS, ADS795WS, ADS830WS, ADS832WS, ADS845MC, ADS870MC, ADS880MC, ADS885MC, ADS890MC, ADS920MC, ADS990MC (American Dye Source, Montreal, Canada); Atto680 (AttoTec, Siegen, Germany); DyLight™ 680 Maleimide, DyLight™ 680 NHS Ester, DyLight™ 800 Maleimide, DyLight™ 800 NHS Ester (Pierce, Rockford, Ill.), DY-680, DY-700, DY-730, DY-750, DY-782 (Dyomics, Jena, Germany); LDS 867, Styryl 15, LDS 925, LDS 950, Phenoxazone 660, Cresyl Violet 670 Perchlorate, Nile Blue 690 Perchlorate, LD 690 Perchlorate, LD 700 Perchlorate, Oxazine 720 Perchlorate, Oxazine 725 Perchlorate, HIDC Iodide, Oxazine 750 Perchlorate, LD 800, DOTC Iodide, DOTC Perchlorate, HITC Perchlorate, HITC Iodide, DTTC Iodide, IR-144, IR-125, IR-143, IR-140, IR-26, DNTPC Perchlorate, DNDTPC Perchlorate, DNXTPC Perchlorate and DMOTC (Exciton, Inc., Dayton, Ohio).

There are also many non-commercial NIR dyes that have been synthesized and may be encapsulated in the current invention to form NIR nanoparticles. Examples of these dyes include bispropylcarboxymethlindocyanine (Bugaj, et al J. Biomed. Opt. 6(2), 122-133 (2001)) and 1,1′-bis-(4-sulfobutyl)indotricarbocyanine-5,5′-dicarboxylic acid diglucamide monosodium salt (Licha K. et al Photochem. Photobiol. 72(3):392-398 (2000)).

There are several ways of making the nanoparticles. For instance, the nanoparticles can be made by in-situ emulsion and suspension polymerization in the presence of the dye. In a typical emulsion polymerization for making NIR nanoparticles in this invention, an NIR dye is dissolved or dispersed in a monomer that is preferably solvent-soluble but not water-soluble; a water-soluble free-radical initiator is dissolved in water to form the aqueous phase; the monomer/dye mix is combined with the aqueous phase and mechanical force such as stirring is applied to disperse the monomer in the aqueous phase to form droplets. Heat may be applied if necessary to start the polymerization. As the polymerization progresses, monomers are converted into polymers, the NIR dye is encapsulated in the resulting polymer and the nanospheres are formed. Optionally, one or more co-monomers are used so that the nanospheres comprise a copolymer. The co-monomers may be either soluble in the solvent or in water. In the case that the co-monomer is water-soluble, it is normally mixed with water for the emulsion polymerization. Examples of said solvent-soluble monomer include styrene, methyl methacrylate, methacrylate, acrylonitrile, vinyl chloride; examples of said water-soluble monomers include acrylic acid and methacrylic acid. The free radical initiator is preferably water-soluble. One example of a water-soluble initiator is ammonium persulfate. Optionally, a surfactant can be used for forming small particles and stabilizing them. Such obtained NIR nanoparticles are separated and purified by centrifuge or dialysis.

Miniemulsion, seeded emulsion, inverse emulsion and microemulsion polymerizations are also suitable for preparing the NIR nanoparticles. Miniemulsions are dispersions of critically stabilized oil droplets with a size between 50 and 500 nm prepared by shearing a system containing oil, water, a surfactant and a hydrophobe. Polymerizations in such miniemulsions, when carefully prepared, result in latex particles which have about the same size as the initial droplets. Said shearing source may be a sonicator, a microfluidizer, or a homogenizer. The details of miniemulsions can be found from Schork F. J. et al, Advances in Polymer Science, Vol. 175, 129-255, 2005.

In preparing NIR nanoparticles of the present invention using miniemulsion, an NIR dye is first dissolved or dispersed in a monomer, typically a chemical containing a polymerizable groups such as a double bond; a hydrophobe such as polystyrene, poly(vinyl acetate), hexadecane are also dissolved in the monomer; a surfactant is dissolved in water and mixed with the monomer solution or suspension; high shear is then applied to form the droplets with desired sizes; a free-radical initiator is used to initialize the polymerization. Said initiator may be either water-soluble or oil-soluble. Ammonium persulfate is a commonly used water-soluble free-radical initiator and azobisisobutylonitrile (AIBN) is a common oil-soluble initiator. Examples of monomers are styrene, methyl methacrylate, butyl acrylate, vinyl chloride, acrylonitrile, etc. Examples of surfactants include sodium dodecyl sulfate (SDS), cetyltrimethylammonium chloride (CTMACI), styrene and ethylene oxide block copolymer (SE3030), and Lutensol AT50 of BASF Corporation. Polymerization may be started with or without heat and typically lasts for several hours. Such obtained NIR nanoparticles are separated and purified by centrifuge or dialysis.

The nanoparticles can also be made by mixing a polymer solution with a dye solution followed by adding a non-solvent to the polymer/dye mixture. Said non-solvent can be an organic solvent, an aqueous solution, or water. The following procedure serves as an example to illustrate how an NIR nanoparticle can be made:
1) dissolving a functionalized polymer in an organic solvent to form solution A;
2) dissolving an NIR dye in an organic solvent to form solution B;
3) mixing solution A and B to form solution C;
4) mixing solution C with a solvent D, said solvent D is preferably selected from the group consisting of water, ethanol, methanol, and isopropyl alcohol;
5) Separating the organic solvents and un-trapped dye molecules from the resulting nanoparticle suspension, and dispersing the nanoparticles in an aqueous media, said aqueous media being distilled or de-ionized water or a buffer solution.

Suitable targeting agents are biomolecules that specifically target certain types of tumors or other biological events. There are many such targeting agents that can be used to form conjugates with the nanoparticles. Suitable targeting agents include peptides, truncated peptides, proteins, hormones, antibodies, antibody fragments, oligonucleotides, small molecules, and mixtures thereof.

In one embodiment of the invention, the targeting agent is a somatostatin (ST) peptide or its analogues. ST is a polypeptide with 13 or 28 amino acid units (ST-14 or ST-28) that bind to ST receptors (STR). ST has shown the growth of tumors by interfering with epidermal growth factors and growth hormone release. Five sub-types (STR1-5) of human ST receptors are well characterized. The expression of STR is high in various tumors (Froidevaus, S. and Eberle, A. N., Biopolymers 66, 161-183, 2002). Smaller ST peptide analogues having longer half-times are more efficient than ST themselves. An example is Octreotide, which is a product of Novartis, Switzerland. OctreoScan® is an ¹¹¹In-DTPA conjugate of octreotide, developed and sold by Mallinckrodt Inc. of Hazelwood, Mo. as an imaging agent that can help find primary and metastatic neuroendocrine tumors. An analogue of octreotide, octreotate, has improved pharmacokinetics and higher STR binding affinity. Octreotide and octreotate are both peptides and can be conjugated to the nanoparticles as optical molecular imaging probes. The nanoparticle-ST peptides conjugates are STR-avid probes useful for imaging breast cancer and other tumors.

In another embodiment of the invention, Bombesin (BN) is conjugated to the NIR nanoparticles. BN is a cell surface receptor protein and is up-regulated in small cell lung, ovarian, pancreatic, colorectal, and prostate cancers. BN has high binding affinity to gastrin-releasing peptide receptor (GRPr). Truncated BN peptide analogues, reported by Scopinaro, F. et al (Eur. J. Nucl. Med. Mol. Imaging 30, 1378-1382, 2003), Okarvi, S. M. et al (Anticancer Res. 23, 2745-2750, 2003), Hoffinan, T. J. et al (J. Nucl. Med. 44, 823-831, 2003), Bajo, A. M. et al (J. Cancer 90, 245-252, 2004), Bajo, A. M. et al (Proc. Natl. Acad. Sci. USA 99, 3836-3841, 2002), Reubi, J. C. et al (Clin. Cancer Res., 8, 1139-1146, 2002), and Karra, S. R. et al (Bioconjugate Chem. 10, 254-260, 1999), have shown improved stability over the native BN and are preferred as the targeting agent in the present invention for forming the nanoparticle-targeting agent conjugates for optical molecular imaging.

In another embodiment of the present invention, the nanoparticles are conjugated to anti-HER2. Her2 is a transmembrane protein belonging to the human epidermal growth factor tyrosine kinase receptor family and is overexpressed in several cancer types, but not in normal tissue. In breast and ovarian cancers, for example, HER2 is overexpressed in 23 to 30% in all cases. American Society of Clinical Oncology recommends detection of HER2 expression in all newly diagnosed or recurrent breast carcinomas in order to select patients who will benefit from treatment with Herceptin and anthracyclines. Orlova, A. et al (Cancer Res. 2006; 66(8), 4339-4348) described a radioactive tumor imaging using a picomolar affinity HER2 binding Affibody® molecule, Z_{HER2-243} (Affibody AB, Bromma Sweden), that showed a >2,200-fold increase in affinity achieved through a single-library affinity maturation step. When radioiodinated, the affinity-matured Affibody® molecule showed high-contrast visualization of HER2-expressing xenografts in mice 6 hours after injection. However, radioactive materials were used in that study. In the current invention, the Affibody® molecule is conjugated to the NIR nanoparticles and the conjugate is suitable for optical molecular imaging for tumor detection. Use of optical method to replace the radioactive one overcomes the side effects of radioactive materials.

In another embodiment of the present invention, the nanoparticles are conjugated to neurotensin (NT) or its truncated analogues. Native NT, truncated NT and their analogues including non-peptide (Feng, H. J. et al Bioorg. Med. Chem., 10, 3849-1858, 2002; Leyton, J. et al Eur. J. Pharmacol. 442, 179-186, 2002), cyclic peptide (Lundquist, J. T. et al Bioorg. Med. Chem. Lett. 9, 2579-2582, 1999), and pseudo-NT peptide (Chavatte, K. et al J. Label. Compd. Radiopharm. 42, 423-435, 1999; Garcia-Garayoa, E. et al Mucl. Med. Biol. 28, 75-84, 2001; Lugrin, D. et al Eur. J. Pharmacol. 205, 191-198, 1991; Gonzalezmuniz, R. et al J. Med. Chem. 28, 1015-1021) can all be conjugated to the nanoparticles in the present invention. The resulting conjugates are particularly useful for imaging pancreatic cancer.

In yet another embodiment of the present invention, the NIR nanoparticles are conjugated to anti-B7-H4 and the like. B7-H4 is a protein of B7 family, and has been found to be highly expressed in ductal and lobular breast cancer (Tringler B. et al Clin. Cancer Res. 11, 1842-1848, 2005). It is also overexpressed in ovarian cancer. The conjugate between the NIR nanoparticles and B7-H4 antibody is particularly suitable for imaging breast and ovarian cancers.

Other targeting agents that may be used to conjugate the NIR nanoparticles in the current invention include the antibodies of CEA, epidermal growth factor receptor (EGFR), hK8, hK14, folate receptors, vasoactive intestinal peptide (VIP) receptor, hydroxyapatite, glucose transporters, EIG121, CDK6, h-Caldesmon , D2-40 and podoplanin, CDX2, minichromosome maintenance protein 5 (Mcm5), and J591.

Said nanoparticle and targeting agent are linked by a covalent or non-covalent bond, but preferably by a covalent bond, formed through the reaction of the functional group on the functionalized polymer surrounding the nanoparticles and the functional group on a biomolecule.

Based on different targeting agent, each type of conjugates is useful for imaging, diagnosis, and prognosis of one or more specific types of cancers or other diseases. The types of cancers the conjugates can be used to detect include but are not limited to breast, ovarian, lung, brain, prostate, colon, stomach, pancreatic and liver cancers.

There are two types of conjugation methods that can be used in the present invention to attach a targeting agent to the nanoparticles: direct conjugation and indirect conjugation. In a direct conjugation method, the reactive groups of the targeting agent are reacted directly with the reactive groups on the surface of the nanoparticle. The common reactive groups include carboxylic, amino, hydroxyl, halide, thiol, isocyanate, aldehyde, the like, and mixtures thereof. For example, the nanoparticles may contain carboxyl groups on their surfaces, and in this case, the carboxyl groups of the nanoparticles react with the amino groups of the targeting agent to form an amide bond between the former and the latter. The carboxyl groups are usually first activated by an activation agent, such as a carbodiimide, the activated carboxyl groups react with amino groups more easily. A common carbodiimide is 1-ethyl-3-(3-dimenthylaminopropyl)carbodiimide, or EDAC.

There are existing protocols for directly conjugating polymer nanoparticles to antibodies, peptides, proteins and other biological molecules. These methods may be modified so that the conjugates of the present invention can be prepared. For different NIR nanoparticles and targeting agents, the conjugation procedures may vary as well. For example, conjugating a carboxylic functional polymer-encapsulated dye nanoparticle with biological molecules method may involve the following steps:
1) dispersing the nanoparticles in a buffer with a pH of 6.0-6.5,
2) dissolving the biomolecule (antibody, protein, peptide, etc.) in the above mentioned buffer,
3) mixing the nanoparticle dispersion and the biomolecule solution,
4) activating the carboxyl groups of the nanoparticles with a carbodiimide such as 1-ethyl-3-(3-dimethylaminopropyl)carbodiimide, and
5) removing unbound biomolecules by centrifuge or dialysis.

### Such obtained conjugates may be then stored in a buffer solution such as phosphate buffer saline.

In an indirect conjugation process, the targeting agent is not directly conjugated to the NIR nanoparticle but to a secondary antibody. The secondary antibody is a species specific antibody. For example, in the case that the targeting agent is a mouse anti-human HER2, the secondary antibody can be a mouse anti-mouse IgG, a rat anti-mouse IgG, a goat anti-mouse IgG, a rabbit anti-mouse IgG, etc. Alternatively, the NIR nanoparticles can also be conjugated to avidin or streptavidin and then linked to a biotinylated secondary antibody through biotin-avidin or biotin-streptavidin reaction.

The nanoparticle-biomolecule conjugates may be applied to a small animal or human body for imaging. Although there are many ways to deliver the conjugates, injection is a common administration method. There are various optical imaging methods that can be used in the present invention. These imaging methods include but are not limited to CCD imaging/spectropolarimeter, confocal microscopy, single- and two-photon microscopy, fluorescence reflectance imaging, diffuse optical tomography, fluorescence molecular tomography, and bioluminescence imaging.

Examples of commercially available optical imaging systems include eXplore Optix of GE Healthcare, Ontario, Canada, KODAK Image Station of Kodak Molecular Imaging, New Haven, Conn., The IVIS® Imaging System of Caliper Life Science, Hopkinton, Mass., and IV100 and OV100 of Olympus America, Center Valley, Pa. The conjugates of the NIR nanoparticles and targeting agents of this invention can be applied to small animals and their fluorescent imaging can be recorded using those imaging systems.

The agents and methods disclosed in the present invention are useful for diagnosis and prognosis of human diseases. They are also useful for in vivo experiments in small animals for the purpose of pre-clinical drug discovery.

The following examples merely illustrate the invention. Those skilled in the art will recognize many variations that are within the spirit of the invention and scope of the claims.

### EXAMPLE 1

### NANOPARTICLES CONTAINING POLY (ACRYLONITRILE-CO-ACRYLIC ACID) ENCAPSULATED indocyanine Green (ICG)

Poly(acrylonitrile-co-acrylic acid), PANA, is synthesized according to S. S. Moghadam et al, Iranian Polym. J. Vol. 14, No. 12, 1032 (2005). Acrylonitrile (90 parts by weight) is mixed with acrylic acid (10 parts by weight); the mixture is added to a 50/50 DMF/water media under nitrogen. The total weight of water and DMF are three times greater than that of the monomers. The copolymerization is carried out at 60° C. for 3 hours. AIBN (2 wt % based on the monomers) is used as the initiator. The yield of the copolymer is approximately 66%. The copolymer obtained is washed with approximately 10 ml of distilled water per gram of copolymer, and dried in an oven at 50° C. overnight. The copolymer is found by NMR to have an acrylic acid molar content of approximately 10%. Intrinsic viscosity, [η], of the copolymer is measured in DMF using an Ubbelohde viscometer in a water bath at 25° C. and is found to be 2.67 dL/g.

Fifty mg PANA prepared as above is dissolved in 5 ml DMSO. 1.5 mg ICG dye (Sigma-Aldrich) is dissolved in 0.75 ml DMSO. The dye solution is then mixed with the polymer solution. To the PANA/dye mixture, 30 ml 1 mM NaOH aqueous solution is added dropwise while stirring. During the addition of the aqueous solution, the nanoparticle suspension is formed. After the addition of the NaOH solution, the resultant nanoparticle suspension is purified by dialysis using a Spectrum dialysis tube (molecular weight cutoff 10,000) in 8 liters of phosphate buffer. The size of the nanoparticles obtained is measured by a Brookhaven Zeta Particle Sizer and is found to be 130 nm.

### EXAMPLE 2

### SYNTHESIS OF NANOPARTICLES CONTAINING POLY(STYRENE-CO-ACRYLIC ACID) ENCAPSULATED ADS760MP BY EMULSION POPOLYMERIZATION

ADS760MP (C₃₉H₄₃Cl N₂O₅) is available from American Dye Source, Inc., 555 Morgan Blvd., Baie D'Urfé, Quebec, H9X 3T6, Canada. A half gram of ADS760MP dye is dissolved in 20 g styrene. Ammonium persulfate (0.1 g) is dissolved in 200 ml de-ionized water. Two grams of acrylic acid is mixed with the initiator solution and combined with the styrene/dye mix. The surfactant-free emulsion copolymerization is carried out at 70° C. for 7 hours with stirring at 350 rpm. The resulting nanoparticles are purified by centrifuging and washing with phosphate buffer three times and stored at 4° C. in phosphate buffer. The size of the nanoparticles obtained is measured by a Brookhaven Zeta Particle Sizer and is found to be 155 nm.

### EXAMPLE 3

### NANOPARTICLES CONTAINING POLY(METHYL METHACRYLATE-CO-METHACRYLIC ACID) ENCAPSULATED IR-140

Poly(methyl methacrylate-co-methacrylic acid), P(MMA-MA), with an MMA/MA ratio of 1:0.16, and IR-140 dye are both from Sigma-Aldrich. One hundred mg PMMA-MA is dissolved in 10 ml DMSO. A half mg IR-140 dye is dissolved in 0.25 ml DMSO. The dye and the polymer solutions are combined. To the polymer/dye mixture, 55 ml 1 mM NaOH aqueous solution is added dropwise while stirring. During the addition of the aqueous solution, the nanoparticle suspension formed. After the addition of NaOH solution, the resulting nanoparticle suspension is purified by dialysis using a Spectrum dialysis tube (molecular weight cutoff 10,000) in 8 liters of phosphate buffer. The size of the nanoparticles obtained is measured by a Brookhaven Zeta Particle Sizer and is found to be 236 nm.

### EXAMPLE 4

### NANOPARTICLES CONTAINING PLGA-COOH ENCAPSULATED INDOCYANINE GREEN (ICG)

PLGA-COOH (LACTEL Absorbable Polymers 50DG020A) with an inherent viscosity of 0.67, a weight average molecular weight of 96,700, and a LA/GA ratio of 50/50, is a product of DURECT Corporation. ICG (cardiogreen) dye is from Sigma-Aldrich. One hundred mg of PLGA-COOH is dissolved in 10 ml tetrahydrofuran (THF). One mg ICG is dissolved in 0.25 ml DMSO and mixed with the PLGA-COOH solution. To the polymer/dye mixture, 55 ml 1 mM NaOH aqueous solution is added dropwise while stirring. During the addition of the aqueous solution, the nanoparticle suspension formed. After the addition of NaOH solution, the resulting nanoparticle suspension is purified by dialysis using a Spectrum dialysis tube (molecular weight cutoff 10,000) in 8 liters of phosphate buffer. The size of the nanoparticles obtained is measured by a Brookhaven Zeta Particle Sizer and is found to be 178 nm.

### EXAMPLE 5

### NANOPARTICLES CONTAINING P(MMA-MA) ENCAPSULATED IR-1048

IR-1048 is from Sigma-Aldrich. One hundred mg P(MMA-MA) is dissolved in 10 ml DMSO. A half mg IR-1048 dye is dissolved in 0.25 ml DMSO. The dye and the polymer solutions are combined. To the polymer/dye mixture, 55 ml 1 mM NaOH aqueous solution is added dropwise while stirring. During the addition of the aqueous solution, the nanoparticle suspension is formed. After the addition of NaOH solution, the resulting nanoparticle suspension is purified by dialysis using a Spectrum dialysis tube (molecular weight cutoff 10,000) in 8 liters of phosphate buffer. The size of the nanoparticles obtained is measured by a Brookhaven Zeta Particle Sizer and is found to be 172 nm.

### EXAMPLE 6

### SYNTHESIS OF CONJUGATES OF PANAA/ICG NANOSPHERES AND ANTI-HER2

Anti-HER2 Affibody® molecule is a product of Affibody, Bromma, Sweden. One ml of 0.5% ICG nanoparticle obtained in Example 1 is centrifuged at 14,000×g for 30 minutes. After the supernatant is decanted, the nanoparticles are re-suspended in 0.5 ml 50 mM MES buffer (pH=6). The nanoparticle suspension is vortexed for 20 seconds, then sonicated in a bath sonicator for 2 minutes. Twenty five μl of freshly prepared 1-ethyl-3-(3-dimenthylaminopropyl)carbodiimide (EDAC) solution in MES buffer (10 mg/ml) is added to the nanoparticle suspension followed by vortexing and sonicating. The mixture is incubated on a shaker for 20 minutes. Another 25 μl of the EDAC solution is added to the reaction mixture followed again by voltexing, sonicating and incubating. The reaction mixture is washed twice with 1 ml 50 mM MES buffer. After centrifuging at 14,000×g for 30 minutes, the nanosphere mixture is suspended in 250 μl of de-ionized water, and then mixed with 250 μl of Anti-HER2 Affibody® solution (400 μg/ml in 100 mM MES buffer). The resultant mixture is incubated on a shaker for 2.5 hours. Ethanolamine (1.25 μl) is added to the reaction mixture and the resultant mixture is incubated for 10 minutes. The reaction mixture is then dialyzed against 150 ml phosphate buffer saline (PBS, pH 7.4) containing 1% BSA and optionally 0.1% NaN₃. The conjugate suspension is stored at 4° C.

### EXAMPLE 7

### CONJUGATES OF PSTAA/ICG NANOSPHERES AND ANTI-EGFR

Anti-EGFR Affibody® is a product of Affibody, Bromma, Sweden. One ml of 0.5% ICG nanoparticle obtained in Example 4 is centrifuged at 14,000×g for 30 minutes. After the supernatant is decanted, the nanoparticles are re-suspended in 0.5 ml 50 mM MES buffer (pH=6). The nanoparticle suspension is vortexed for 20 seconds and then sonicated in a bath sonicator for 2 minutes. Freshly prepared 1-ethyl-3-(3-dimenthylaminopropyl)carbodiimide (EDAC) solution (25 μl) in MES buffer (10 mg/ml) is added to the nanoparticle suspension followed by vortexing and sonicating. The mixture is incubated on a shaker for 20 minutes. Another 25 μl of the EDAC solution is added to the reaction mixture followed again by voltexing, sonicating and incubating. The reaction mixture is washed twice with 1 ml 50 mM MES buffer. After centrifuging at 14,000×g for 30 minutes, the nanosphere mix is suspended in 250 μl of de-ionized water, and then mixed with 250 μl of Anti-EGFR Affibody® solution (400 μg/ml in 100 mM MES buffer). The resultant mixture is incubated on a shaker for 2.5 hours. Ethanolamine (1.25 μl) is added to the reaction mixture and the resultant mixture is incubated for 10 minutes. The reaction mix is then dialyzed against 150 ml phosphate buffer saline (PBS, pH 7.4) containing 1% BSA and optionally 0.1% NaN₃. The conjugate suspension is stored at 4° C.

I claim:

1. An image probing conjugate comprising a nanoparticle and a targeting agent, wherein the nanoparticle comprises a dye which is encapsulated by a functionalized polymer, and wherein the targeting agent is bound to the functionalized polymer through a functional group.

2. The conjugate of claim 1, wherein the targeting agent is covalently bound to the functionalized polymer through a functional group.

3. The conjugate of claim 1, wherein the functional group is selected from the group consisting of carboxylic, amino, hydroxyl, halide, thiol, isocyanate, aldehyde, and mixtures thereof.

4. The conjugate of claim 1, wherein the functional group is a carboxyl group.

5. The conjugate of claim 4, wherein the carboxylic functional polymer is selected from the group consisting of poly(acrylonitrile-co-acrylic acid), poly(acrylonitrile-co-methacrylic acid), poly(styrene-co-acrylic acid), poly(styrene-co-methacrylic acid), poly(methyl methacrylate-co-acrylic acid), poly(methyl methacrylate-co-methacrylic acid), poly(lactic acid), poly(glycolic acid), poly(lactic acid-co-glycolic acid), polycaprolactone, poly(lactic acid-co-caprolactone), and mixtures thereof.

6. The conjugate of claim 1, wherein the dye is a near-infrared dye and has an excitation peak of 650-1200 nm.

7. The conjugate of claim 6, wherein the near-infrared dye is selected from the group consisting of indocyanine green, IR-780 iodide, IR-780 perchlorate, IR-27, IR-140, IR-676, IR-676 iodide, IR-746, IR-768 perchlorate, IR-775 chloride, IR-777 perchlorate, IR-780 iodide, IR-780 perchlorate, IR-783, IR 786 iodide, IR-786 perchlorate, IR-792 perchlorate, IR-797 chloride, IR-797 perchlorate, IR-806, IR-813 chloride, IR-813 perchlorate, IR-820, and mixtures thereof.

8. The conjugate of claim 1, wherein the dye is indocyanine green.

9. The conjugate of claim 1, wherein the nanoparticle has an average particle size of less than or equal to 1,000 nm.

10. The conjugate of claim 1, wherein the nanoparticle has an average particle size of less than or equal to 500 nm.

11. The conjugate of claim 1, wherein the targeting agent is selected from the group consisting of peptides, truncated peptides, proteins, hormones, antibodies, antibody fragments, oligonucleotides, small molecules, and mixtures thereof.

12. A method of preparing an image probing conjugate, said method comprising:
(a) producing a nanoparticle by encapsulating a dye in a functionalized polymer; and
(b) mixing the nanoparticle with a targeting agent and bonding the targeting agent to the functional group of the nanoparticle.

13. The method of claim 12, wherein the nanoparticle is produced by mixing the functionalized polymer and the dye in a solution, encapsulating the dye with the polymer, and precipitating the polymer-encapsulated dye from the solution.

14. The method of claim 12, wherein the nanoparticle is produced by in-situ emulsion or suspension polymerization.

15. The method of claim 12, wherein the functionalized polymer has a functional group selected from the group consisting of carboxylic, amino, hydroxyl, thiol, isocyanate, aldehyde, and mixtures thereof.

16. The method of claim 12, wherein the ftinctionalized polymer is a carboxylic functional polymer selected from the group consisting of poly(acrylonitrile-co-acrylic acid), poly(acrylonitrile-co-methacrylic acid), poly(styrene-co-acrylic acid), poly(styrene-co-methacrylic acid), poly(methyl methacrylate-co-acrylic acid), poly(methyl methacrylate-co-methacrylic acid), poly(lactic acid), poly(glycolic acid), poly(lactic acid-co-glycolic acid), polycaprolactone, poly(lactic acid-co-caprolactone), and mixtures thereof.

17. The method of claim 12, wherein the dye is a near-infrared dye selected from the group consisting of indocyanine green, IR-780 iodide, IR-780 perchlorate, IR-27, IR-140, IR-676, IR-676 iodide, IR-746, IR-768 perchlorate, IR-775 chloride, IR-777 perchlorate, IR-780 iodide, IR-780 perchlorate, IR-783, IR 786 iodide, IR-786 perchlorate, IR-792 perchlorate, IR-797 chloride, IR-797 perchlorate, IR-806, IR-813 chloride, IR-813 perchlorate, IR-820, and mixtures thereof.

18. The method of claim 12, wherein the nanoparticle has an average particle size less than or equal to about 1,000 nm.

19. The method of claim 12, wherein the targeting agent is selected from the group consisting of peptides, truncated peptides, proteins, hormones, antibodies, antibody fragments, oligonucleotides, small molecules, and mixtures thereof.

20. A method of in vivo optical imaging, comprising:
(a) administering to a human or animal body an optical imaging probe conjugate that comprises a particulate and a targeting agent, wherein the particulate is a nanoparticle comprising a dye encapsulated by a functionalized polymer, and wherein the targeting agent is bound to the functional group;
(b) waiting for the optical imaging probe to reach the target tissue;
(c) illuminating the target tissue with light of a wavelength absorbable by the optical imaging probe; and
(d) detecting the optical signal emitted by the probe.
